(19) United States
(12) Patent Application Publication
(10) Pub. No.: US 2022/0355045 A1
(43) Pub. Date: Nov. 10, 2022

(54) HAIR GROWTH APPLICATOR AND METHOD OF USE

(71) Applicant: Michael Berry, Woodmere, NY (US)

(72) Inventor: Michael Berry, Woodmere, NY (US)

(21) Appl. No.: 17/732,221

(22) Filed: Apr. 28, 2022

Related U.S. Application Data
(60) Provisional application No. 63/183,898, filed on May 4, 2021.

(51) Int. Cl. A61M 5/32; A61F 13/40; A61M 5/178

(52) U.S. Cl. CPC A61M 5/3298; A61M 35/006; A61M 5/1782; A61M 2210/06

(57) ABSTRACT

A hair growth applicator for promoting the growth of hair follicles by applying hair growth liquid, comprising a handle having an internal chamber for containing said hair growth liquid, said handle having a channel running from said chamber to a distal end of said handle, a tip disposed at said distal end of said handle with a plurality of needles disposed on said distal surface of said tip, said distal surface having an opening, wherein said chamber is in fluid connection with said opening on said distal surface of said tip

### PRIOR APPLICATION

This non-provisional patent application claims priority to U.S. Provisional Application No. 63/183,898, filed on May 4, 2021 and incorporated herein by reference in its entirety.

### TECHNICAL FIELD

The present disclosure relates generally to a hair growth applicator and method of use, and more particularly to a hair growth applicator having a multitude of needles for penetrating the epidermis of the scalp to allow a hair growth liquid more direct contact with treatment sites.

### BACKGROUND OF THE INVENTION

Pattern hair loss is hair loss that primarily effects the top and front of the scalp. Pattern hair loss, while quite common, can be a stressful condition and often diminishes body image satisfaction. As a result, many people suffering from pattern hair loss choose to take steps to prevent further hair loss and promote new hair growth.

Many of the programs designed to fight pattern hair loss include the use of minoxidil and/or finasteride. Minoxidil is typically applied topically to the site of hair loss to promote hair growth. While topically applying Minoxidil by simply rubbing a Minoxidil-based substance (e.g. a cream or ointment) onto the scalp has been shown to be somewhat effective in fighting the effects of pattern hair loss, there are many individuals that do not achieve the results they desire with ordinary topical application. One issue with applying a Minoxidil-based cream on the surface of the skin is that the actual source of hair follicle growth lies below the surface of the skin. Therefore, applying a Minoxidil-based cream on the surface of the skin may not allow the cream to penetrate deep enough into the user's scalp for optimal results.

Aspects of the present disclosure are directed to overcoming these and other problems.

### SUMMARY OF THE INVENTION

The present disclosure relates to a hair growth applicator, which can be used together with a hair growth liquid (and/or creams) to treat pattern hair loss including treating baldness, promoting hair growth, and/or promoting hair follicle development and/or activation on an area of the skin of a subject (e.g. the scalp of a person). The hair growth applicator includes small needles disposed on the tip of the applicator, which penetrate the outer skin of the user's scalp to allow a hair growth liquid (and/or creams) to contact treatment sites on the user's scalp that lie below the outer skin.

The hair growth applicator and method of using the applicator can be used in combination with other treatments for promoting hair growth. The present disclosure also relates to various pharmaceutical compositions for the hair growth liquid (and/or creams) that may increase the efficacy of the hair growth applicator and the method of using the same to promote hair growth.

### BRIEF DESCRIPTION OF THE DRAWINGS

FIG. 1 is a plan view of a hair growth applicator in accordance with the present disclosure.

FIG. 2 is an exploded view of the hair growth applicator of FIG. 1, and in accordance with the present disclosure.

FIG. 3 is a detailed, close-up view of the tip of the hair growth applicator of FIG. 1, and in accordance with the present disclosure.

FIG. 4 is perspective view of the hair growth applicator of FIG. 1 being used to treat a bald spot on a person's scalp.

FIG. 5 is a method of using the hair growth applicator of FIG. 1.

### DETAILED DESCRIPTION OF THE INVENTION

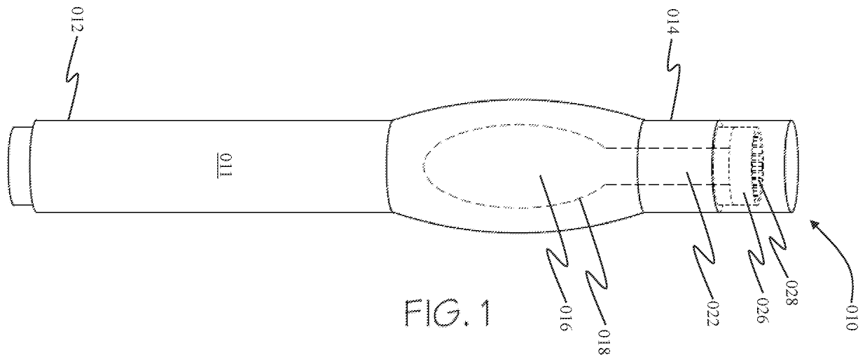

FIG. 1 is a hair growth applicator 010 in accordance with examples described herein. FIG. 1 illustrates a handle 011, a tip 026, and a cap 050.

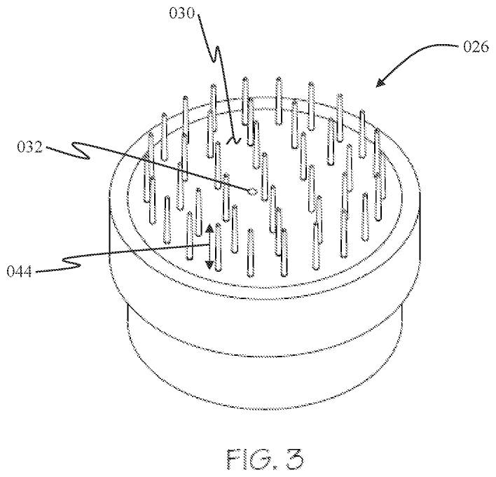

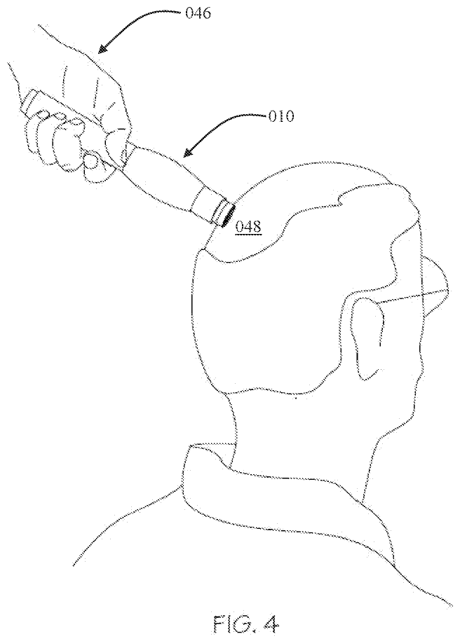

Referring to FIGS. 1-4, handle 011 has a proximal end 012 and a distal end 014. Handle 011 includes a chamber 016 comprising an open space for containing a hair growth liquid or cream or similar substance to be applied topically to a user. Handle 011 includes a channel 022 running from the chamber 016 to the distal end 014 of handle 011. In a preferred embodiment, handle 011 is pliable and/or elastic (at least in the area of the handle walls proximal to the chamber 016) such that a user 046 can squeeze handle 011 in the area proximal to the chamber 016 to cause the chamber walls 018 to bow inward and thereby cause the liquid in the chamber 016 to flow or extrude out of chamber 016 and through channel 022 toward distal end 014 of handle 011. At distal end 014 of handle 011 is a fastening mechanism 024 such that handle 011 is adapted to be mated with tip 026 via an interference fit or other type of suitable connectivity. In another embodiment, tip 026 utilizes a fastening mechanism that is permanent (e.g. in a disposable version of the product that does not need to be refilled). In a further embodiment, tip 026 is removably fastened other than by an interference fit, such as by threading together, latching via a camming mechanism, or any other suitable fastening known to those of skill in the art having the benefit of the present disclosure.

Referring to FIGS. 1-4, tip 026 is disposed on the distal end 014 of handle 011. The tip 026 has a series of needles 028 disposed on a distal surface 030 of the tip 026. The distal surface 030 of the tip 026 also has an opening 032 bored through an outer wall of the tip 026, extending from an interior surface of the outer wall to an exterior distal surface 030 of the outer wall, such that hair growth liquid can pass through the opening 032 from the inner cavity 040 of the tip 026 to the distal surface 030 of the outer wall of the tip. The needles 028 are embedded in the outer wall of the tip 026 such that the needle extension 044 (i.e. distance between the distal surface 030 of the tip 026 and the distal point of each of the needles 028) is approximately 0.05 mm. In other embodiments, the needle extension 044 may be in the range of 0.01 mm-0.08 mm, and more preferably in the range of 0.03 mm-0.07 mm.

In another preferred embodiment, a tip with a multi-needle array, each of the type used for example to deliver insulin or growth hormone, may be utilized to apply hair growth liquid to a user's scalp. In such an embodiment, the tip does not contain a bore through which the hair growth liquid is applied. Rather, the hair growth liquid would be applied directly via the hollow needle ends and openings thereof which are presented in an array on the tip. Notably the same number of needles may be utilized to apply the head growth liquid.

Referring to FIGS. 1-4, tip 026 is configured to be removably fastened to handle 011 through an interference fit such that a user 046 can remove the tip 026 to fill the handle 011 with hair growth liquid and then reinsert the tip 026 into the handle 011 such that the tip 026 is secured from falling out during use.

Referring to FIGS. 1-4, during use of the hair growth applicator 010, a user 046 squeezes the handle 011, which will in turn cause the chamber walls 018 to bow inward and thereby increase the pressure of the liquid within the chamber 016. The increased pressure of the liquid in the chamber 016 will cause the liquid in the chamber 016 to flow or extrude out of the chamber 016 and through the channel 022 toward the distal end 014 of the handle 011. The liquid will then encounter the interior surface of the outer wall of the tip 026. As the user 046 continues to squeeze the handle 011, the pressure of the liquid will continue to increase and cause the liquid to extrude out of the inner cavity 040 of the tip 026, through the opening 032 in the tip 026, and onto the distal surface 030 of the tip 026. The hair growth liquid will then coat the needles 028 of the tip 026 as it spreads across the distal surface 030 of the tip 026. Once the needles 028 are sufficiently coated, the user 046 will then press the tip 026 of the hair growth applicator 010 against the treatment site 048 (e.g. a bald spot on the scalp) and continue to press the tip 026 against the surface of the patient's skin until the needles 028 penetrate the outer skin of the patient's scalp, and thereby deliver the hair growth liquid directly to treatment sites 048 below the surface of the skin. In other embodiments, the hair growth liquid is deposited on the surface of the patient's skin before the needles 028 are inserted into the skin in the area where the hair growth liquid has been deposited, such that the needles 028 enable for deeper penetration of the liquid.

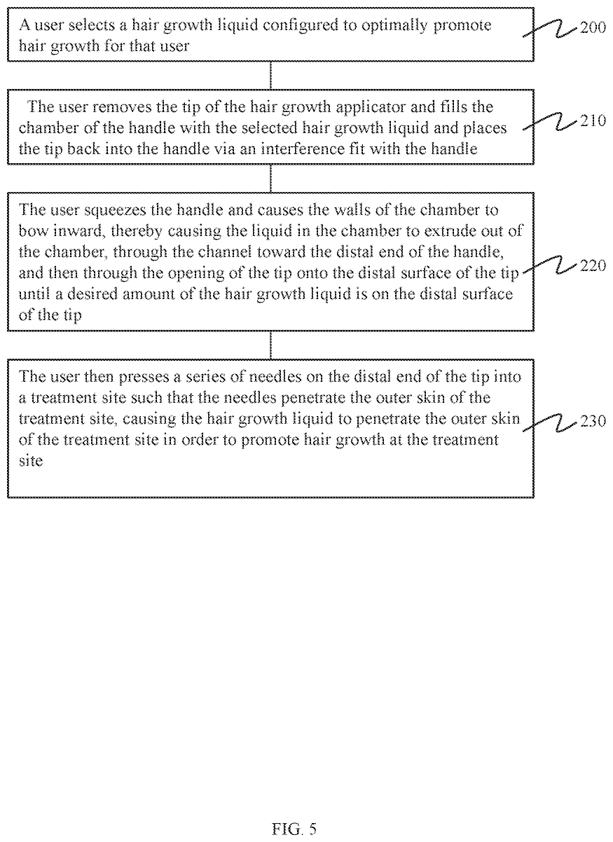

Referring to FIG. 5, a method for using a hair growth applicator as described herein is shown. A user selects a hair growth liquid configured to optimally promote hair growth for that user 200. The user removes the tip of the hair growth applicator and fills the chamber of the handle with the selected hair growth liquid and places the tip back into the handle via an interference fit with the handle 210. The user squeezes the handle and causes the walls of the chamber to bow inward, thereby causing the liquid in the chamber to extrude out of the chamber, through the channel toward the distal end of the handle, and then through the opening of the tip onto the distal surface of the tip until a desired amount of the hair growth liquid is on the distal surface of the tip 220. The user then presses a series of needles on the distal end of the tip into a treatment site such that the needles penetrate the outer skin of the treatment site, causing the hair growth liquid to penetrate the outer skin of the treatment site in order to promote hair growth at the treatment site 230. In other embodiments, the method can be performed in a different order such that the user presses the needles into the treatment site first, and then squeezes the handle and extrudes the hair growth liquid onto the treatment site.

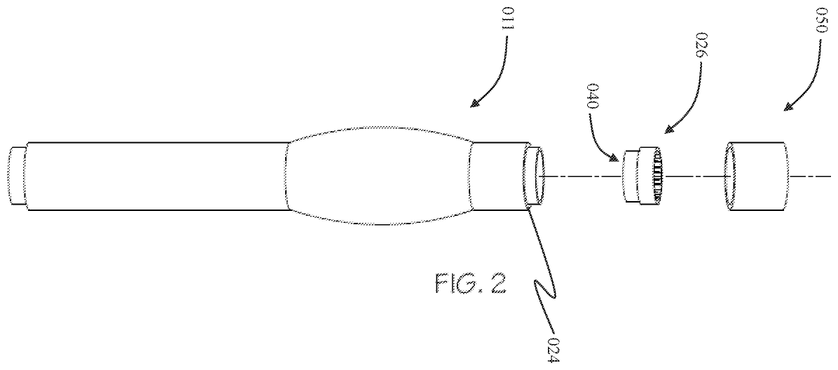

Referring to FIGS. 1-2, a cap 050 can be used to cover the tip 026 of the hair growth applicator 010 when it is not in use. The cap 050 can be attached via an interference fit with the distal end 014 of the tip 026, or by any other means known by those of skill in the art having the benefit of this disclosure.

Referring to FIGS. 1-4, tip 026 as shown is made of a polymer material. In other embodiments, the tip 026 can be made of wood, rubber, silicon, metal, or any other material known by those of skill in the art having the benefit of the present disclosure. In other embodiments, the distal surface 030 of the tip 026 can have a sponge layer disposed on the surface, configured to retain the hair growth liquid as it is extruded out through the opening 032. The sponge layer can be a genuine sponge, synthetic sponge, or any other material that has the property of retaining liquid as known by those of skill in the art. In these embodiments having a sponge layer, the sponge layer serves to absorb the liquid such that it is not deposited onto the treatment site 048 until the needles 028 have fully penetrated the treatment site 048 and the sponge layer is then pressed against the surface of the treatment site 048.

As used herein, hair growth liquid refers to a substance that contains minoxidil and that promotes hair growth. In other embodiments, the hair growth liquid is fortified or mixed with vitamins and compounds to enhance the efficacy of the hair growth liquid. For example, the hair growth liquid might contain vitamin A, biotin, and/or moisturizing compounds to moisturize the skin. In yet other embodiments, the hair growth liquid contains finasteride either in place of or in addition to minoxidil.

In other embodiments, the hair growth applicator 010 may have two tips. A first tip is disposed on a first end of the handle 011 and has a series of needles 028 for penetrating the surface skin of a treatment site 048. The second tip is disposed on a second end of the handle 011 and has an opening 032. The hair growth liquid in the chamber 016 runs through the channel 022 and out of the opening 032 in the second tip. This embodiment allows a user 046 to apply the hair growth liquid separately from the use of the needles 028 to penetrate the surface of the skin of a patient. This embodiment can be used by either applying the hair growth liquid with the second tip first, and then using the needles 028 to penetrate the skin of the treatment site 048 using the first tip, or vice versa.

Example advantages of systems described herein may include an increase in the speed and efficiency of promoting hair growth through use of hair growth liquid, for example, Minoxidil. Specifically, the hair growth applicator 010 allows a substance like liquid Minoxidil to be delivered directly to the root of a hair follicle or potential hair follicle rather than being deposited on the surface of a patient's skin as see in the prior art. Note that the advantages described herein are provided to facilitate an appreciation of examples of the described technology. It is to be understood that examples of the described technology may not exhibit all, or even any, of the described advantages.

As described herein, the hair growth applicator 010 can be used either by a user 046 that is treating a spot on his/her own body or scalp, or by a user 046 to treat a spot on another's body or scalp. Therefore, any reference or language describing either an instance of self-treatment or an instance of treating another is not intended to be limiting.

From the foregoing it will be appreciated that, although specific embodiments have been described herein for purposes of illustration, various modifications may be made while remaining within the scope of the claimed technology.

1. A hair growth applicator for promoting the growth of hair follicles by applying hair growth liquid, comprising:
a handle having an internal chamber for containing said hair growth liquid, said handle having a channel running from said chamber to a distal end of said handle;
a tip disposed at said distal end of said handle, said tip having a distal surface and a plurality of needles disposed on said distal surface of said tip, said distal surface having an opening;
wherein said chamber is in fluid connection with said opening on said distal surface of said tip; and
wherein said plurality of needles are configured to penetrate an exterior surface of skin of a scalp to enable hair growth liquid flowing out of said chamber to contact treatment sites on said scalp below said exterior surface of skin.

2. The hair growth applicator of claim 1, said handle comprising a chamber wall that is elastic and flexible and which is adapted to bow inward in response to an applied force to drive a hair growth compound in said chamber to flow out of said opening.

3. The hair growth applicator of claim 2, said tip being in permanent connection with said handle.

4. The hair growth applicator of claim 2, said tip being in removable fastening with said handle to enable refilling of said chamber with said hair growth liquid.

5. The hair growth applicator of claim 1 having a needle extension distance between said distal surface of said tip and a distal point of each of said plurality of said needles of approximately 0.05 mm.

6. The hair growth applicator of claim 1 having a needle extension distance between said distal surface of said tip and a distal point of each of said plurality of said needles in the range of 0.01 mm to 0.08 mm.

7. The hair growth applicator of claim 6 having a needle extension distance between said distal surface of said tip and a distal point of each of said plurality of said needles in the range of 0.03 mm to 0.07 mm.

8. The hair growth applicator of claim 1, said tip comprising a sponge layer disposed on a surface of said tip and configured to retain said hair growth fluid as it flows through said opening.

9. The hair growth applicator of claim 8, wherein said sponge layer is sized and adapted to absorb said hair growth liquid when it flows from said opening, and is further sized and adapted so that it is not deposited onto skin until said plurality of needles have fully penetrated and said sponge layer is then pressed against said skin for treatment.

10. The hair growth applicator of claim 1 further comprising a cap to cover said opening and said plurality of needles.

11. A method for using a hair growth applicator filled with hair growth liquid comprising the following steps:
providing a hair growth applicator with a handle, an internal chamber interior of said handle for housing hair growth liquid, a tip disposed at a distal end of said handle, said tip having a distal surface and a plurality of needles disposed on said distal surface of said tip, said distal surface having an opening in fluid communication with said interior chamber;
applying a force to drive said hair growth liquid from said chamber to an exterior of said opening until a desired amount of the hair growth liquid is on the distal surface of the tip;
pressing said plurality of needles on said distal end of said tip into a treatment site such that said needles penetrate the outer skin of the treatment site; and
directing said hair growth liquid to treatment site.

12. The method of claim 11, wherein said chamber of said hair growth applicator comprises walls that are flexible and the step of pressing said walls of said chamber to drive liquid from said chamber to an exterior of said opening

13. The method of claim 11, further comprising the step of refilling said chamber of said hair growth applicator with hair growth liquid after use of said hair growth applicator.

14. The method of claim 11, wherein said hair growth applicator has a needle extension distance between said distal surface of said tip and a distal point of each of said plurality of said needles of approximately 0.05 mm.

15. The method of claim 14, wherein said hair growth applicator has a needle extension distance between said distal surface of said tip and a distal point of each of said plurality of said needles in the range of 0.01 mm to 0.08 mm.

16. The method of claim 15, wherein said hair growth applicator has a needle extension distance between said distal surface of said tip and a distal point of each of said plurality of said needles in the range of 0.03 mm to 0.07 mm.

17. A hair growth applicator for promoting the growth of hair follicles by applying hair growth liquid, comprising:
a handle having an internal chamber for containing said hair growth liquid, said handle having a channel running from said chamber to a distal end of said handle;
a tip disposed at said distal end of said handle, said tip having a distal surface and a plurality of needles disposed on said distal surface of said tip, wherein said plurality of said needles are adapted to apply and deliver hair growth liquid via openings at ends of said needles;
wherein said chamber is in fluid connection with said openings at said ends of said needles; and
wherein said plurality of needles are sized and configured to penetrate an exterior surface of skin of a scalp to enable hair growth liquid flowing out of said chamber to contact treatment sites on said scalp below said exterior surface of skin.

18. The hair growth applicator of claim 17, said tip being in permanent connection with said handle.

19. The hair growth applicator of claim 17, said tip being in removable fastening with said handle to enable refilling of said chamber with said hair growth liquid.

20. The hair growth applicator of claim 17 having a needle extension distance between said distal surface of said tip and a distal point of each of said plurality of said needles in the range of 0.01 mm to 0.08 mm.
